(19) United States
(12) Patent Application Publication
(10) Pub. No.: US 2020/0027529 A1
(43) Pub. Date: Jan. 23, 2020

(54) METHOD FOR DETERMINING AFFINITY OF A BIOMOLECULE

(71) Applicant: GE HEALTHCARE BIO-SCIENCES AB, Uppsala (SE)

(72) Inventors: Tomoya Mitani, Shinjuku-ku (JP); Robert Karlsson, Uppsala (SE); Olof Karlsson, Uppsala (SE)

(21) Appl. No.: 15/754,960

(22) PCT Filed: Aug. 25, 2016
(86) PCT No.: PCT/EP2016/070119

§ 371 Date: Feb. 23, 2018

(30) Foreign Application Priority Data
Aug. 25, 2015 (GB) 1515070.9

(51) Int. Cl. G16C 20/30; G01N 33/557; G16C 20/50

(52) U.S. Cl. CPC G16C 20/30; G16C 20/50; G01N 33/557

(57) ABSTRACT

Disclosed is a method for determining affinity of a biomolecule, including the steps of: determining a theoretical saturation response value (Rmax) based on known properties of the biomolecule or a similar biomolecule; providing a sensor surface having the biomolecule immobilized thereon as a ligand; contacting the sensor surface with a plurality of samples containing different concentrations of an analyte that is able to bind to the ligand; registering an equilibrium response (R) from binding of the analyte to binding sites of the ligand for each of the plurality of samples, said sensor response giving equilibrium response values for each sample; creating an equilibrium response sequence comprising the determined equilibrium response values; determining a plurality of values for a dissociation constant for the plurality of concentrations based on said equilibrium response sequence; and determining a theoretical dissociation constant at zero concentration based on the plurality of values for the dissociation constant.

### CROSS REFERENCE

This application is a filing under 35 U.S.C. 371 of international application number PCT/EP2016/0070119, filed Aug. 25, 2016, which claims priority to Great Britain application number 1515070.9, filed Aug. 25, 2015, the entire disclosure of each of which is hereby incorporated by reference.

### TECHNICAL FIELD

The present invention relates to a method for determining affinity of a biomolecule.

### BACKGROUND

Analytical sensor systems that can be used to determine properties of biomolecules are used in a variety of different fields including but not limited to pharmaceutical research and development of new medicines. Such systems may use surface plasmon resonance (SPR), where biomolecules are immobilized on a sensor surface to act as a ligand and a sample containing a known concentration of an analyte is passed over the sensor surface. The interaction between ligand and analyst is detected by the sensor and a plurality of interaction parameters can be determined, among them the affinity of the biomolecules. Other technologies that can be used for determining interaction parameters such as the affinity include calorimetry, thermophoresis and several other technologies capable of generating dose-response curves.

The affinity can be expressed as an association constant (KA) or a dissociation constant (KD) and describes the ability of a binding site on the biomolecule to bind to the analyte. However, depending on the orientation of the biomolecules on the sensor surface, apart from the main binding site one or more secondary binding sites for the analyte may exist, and may be associated with different affinities. It is also possible that the analyte binds to the immobilized molecule promiscuously (e.g. hydrophobic surface of protein) or sensor surface directly, avoiding the specific binding sites of the ligand. To correctly determine low affinities high concentrations are required. For these measurements the often unknown properties of the different binding sites have to be taken into account. This makes it difficult to arrive at accurate measurements and assessments within this area.

There is therefore a need for an improved method for determining affinity of a biomolecule that does not suffer from these drawbacks.

### DISCLOSURE OF THE INVENTION

The object of the present invention is to eliminate or at least to minimize the problems described above. This is achieved through a method for determining affinity of a biomolecule according to the appended independent claim, where a theoretical dissociation constant at zero concentration is determined. Thereby, the effects of secondary binding sites can be minimized and a reliable value for the affinity achieved regardless of the behaviour of the analyte at higher concentrations.

According to one aspect of the invention, a plurality of values for a dissociation constant are iteratively determined until a stable value for the dissociation constant is obtained. The analysis is performed by

determining the dissociation constant corresponding to the highest concentration of the equilibrium response sequence based on the equilibrium response sequence,

selecting the equilibrium response value of the equilibrium response sequence corresponding to the highest concentration and removing said equilibrium response value,

determining the dissociation constant corresponding to the highest remaining concentration of the equilibrium response sequence and again removing the value corresponding to the highest concentration, and

repeating the process of determining the dissociation constant and removing a value from the equilibrium response sequence until the difference between the determined dissociation constant and the dissociation constant determined in the latest repetition is smaller than a convergence value, and

selecting the most recently determined dissociation constant as the theoretical dissociation constant at zero concentration.

Thereby, calculations can be iterated until the value of the dissociation constant converges towards a stable value at very low concentrations, and this value can be taken as the affinity at zero concentration.

According to another aspect of the invention, a plurality of values for a dissociation constant are determined and the theoretical dissociation constant at zero concentration is obtained through extrapolation. The analysis is performed by

determining the dissociation constant corresponding to the highest concentration of the equilibrium response sequence based on the equilibrium response sequence,

selecting the equilibrium response value of the equilibrium response sequence corresponding to the highest concentration and removing said equilibrium response value,

determining the dissociation constant corresponding to the highest remaining concentration of the equilibrium response sequence and again removing the value corresponding to the highest concentration, and

repeating the process of determining dissociation constant and removing a value from the equilibrium response sequence until less than three values remain in the equilibrium response sequence,

plotting each determined dissociation constant against the highest concentration used in the determination of the dissociation constant to create a plurality of data points and fitting said data points to a curve, and

selecting a value along said curve corresponding to zero concentration as the theoretical dissociation constant at zero concentration.

Using curve fitting, a reliable value for the dissociation constant and thereby the affinity at zero concentration can be achieved.

According to yet another aspect of the invention, each of the plurality of values for dissociation constant is determined by selecting a value from the equilibrium response sequence, determining an interval on each side of the value and determining the dissociation constant based on said value and said interval. Thereby, as an alternative to using every value of the sequence and removing them one by one, reliable values for the dissociation constant can be achieved using only one value and a vicinity of said value at a time, thus minimizing the effect of outliers among the values of the equilibrium response sequence.

According to the invention, the method is preferably realized through software configured to perform the method according to the claims and a computer readable medium configured to store said software. Thereby, the steps of the method can be performed in an automated and reliable way without the need for manual input along the way, and the results can be presented in a suitable manner to increase the uses of the invention.

Many more advantages and benefits of the invention will become readily apparent from the detailed description below.

### DRAWINGS

The invention will now be described in more detail with reference to the appended drawings, wherein:

FIG. 1 shows a schematic view of the steps of the method according to the invention;

FIG. 2 shows a diagram of sensor response over time at different concentrations according to one step of the method of FIG. 1;

FIG. 3 shows the sensor responses of FIG. 2 at different concentrations; and

FIG. 4 shows affinity expressed as dissociation constant (KD) at different concentrations according to a step of the method of FIG. 1.

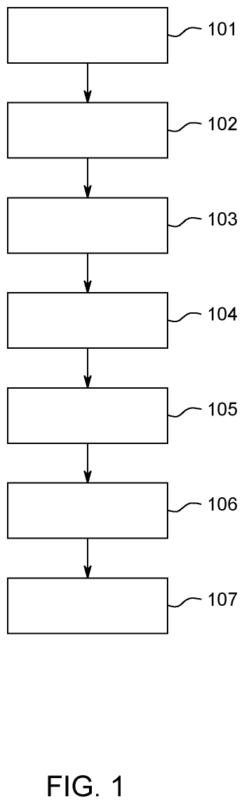

### DETAILED DESCRIPTION

Typically, in order to determine affinity of a biomolecule, experiments are performed using a sensor-based technology such as Surface Plasmon Resonance (SPR). According to this technology, a biomolecule to be analysed, known as a ligand, is introduced to a sensor surface and immobilized thereon. A solution containing a known concentration of another molecule known as an analyte that is able to bind to the ligand is supplied, and properties at the sensor surface change depending on how much of the ligand that binds to the analyte. Thus, a sensor response from the sensor surface shows the interaction between ligand and analyte and can be analysed to yield information such as the affinity of the ligand. In the following, it is assumed that the sensor-based technology used with the invention is SPR, but it is to be noted that other, similar label free methods and solution based methods such as thermophoresis or calorimetry may also be used.

The relationship between detector response (expressed as Req, the detector response at equilibrium), concentration (C), equilibrium response (Rmax) and dissociation constant (KD) can be expressed as:

Req=(Rmax1×C)/(C+KD1)+(Rmax2×C)/(C+KD2)+ . . . Eq 1

where each binding site on the ligand generates one term and has its own equilibrium response and affinity.

Combining the terms for secondary binding sites gives the simplified expression:

Req=(Rmax1×C)/(C+KD1)+X Eq 2

At higher concentrations, the contributions of the secondary binding sites to the detector response increases, making the separation of the terms very difficult. At low concentrations, however, the first term is significantly larger and the rest term X decreases towards 0. By deciding on a fixed equilibrium response R or Rmax, KD can thereby be found and associated with the equilibrium response value R.

Thus, according to a preferred embodiment of the present invention, the dissociation constant KD for the main binding site can be accurately determined by determining theoretically a value for the dissociation constant KD at zero concentration. The method according to this preferred embodiment will now be described with reference to the Figures.

In a first step 101 according to the preferred embodiment of the invention, a theoretical saturation response value Rmax is determined based on known properties of the biomolecule or a similar biomolecule, showing theoretically a situation where every biomolecule of the ligand binds to the analyte. This value can be determined from known, theoretical or experimental data regarding the biomolecule in question but can also be based on known properties of similar molecules. One such method is shown by WO 2011/065913 and other methods are also well-known in the art.

In a second step 102, the sensor surface of the sensor technology is provided and the biomolecule is immobilized as a ligand on said surface. In a third step 103, a dilution series is created by contacting the sensor surface with a plurality of samples containing different concentrations of an analyte that is able to bind to the ligand.

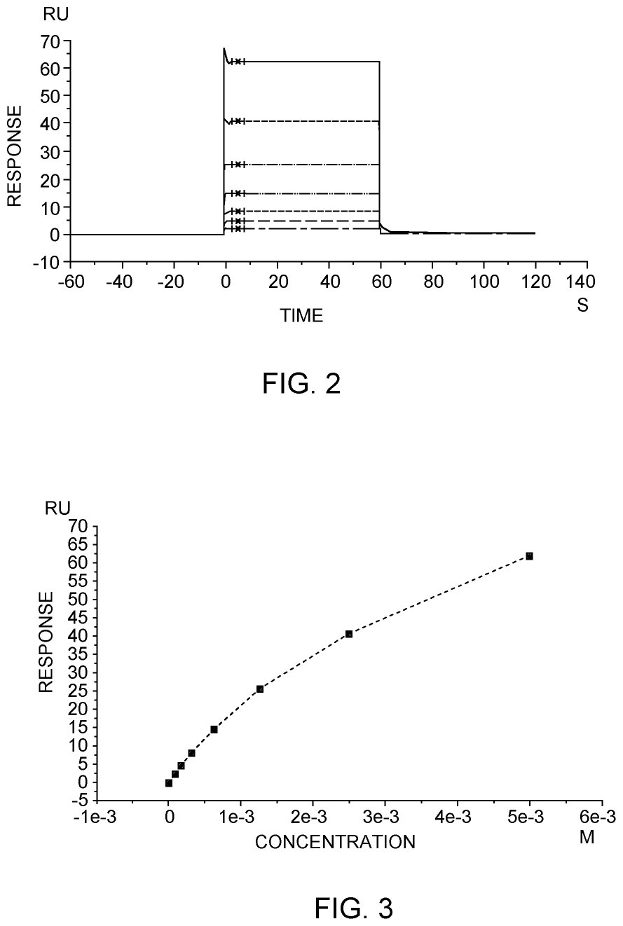

The resulting data is collected in a fourth step 104 by registering a sensor response in the form of an equilibrium response R from binding of the analyte to binding sites of the ligand for each of the plurality of samples, and can be shown in the form of graphs (see FIG. 2), showing the sensor response R over time for each sample. The sensor response registered thereby forms equilibrium response values for each sample.

In a fifth step 105, an equilibrium response value R for each concentration of the plurality of samples are used to create an equilibrium response sequence Rseq comprising the determined equilibrium response values R. The equilibrium response values R are displayed in FIG. 3 and will be further explained below with reference to an example of carrying out the invention using software to perform calculations, curve fittings and determination of theoretical values.

In a sixth step 106, a plurality of values for a dissociation constant KD are determined for the plurality of concentrations based on said equilibrium response sequence Rseq and in a seventh step 107 a theoretical dissociation constant at zero concentration KD0 is determined based on the plurality of values for the dissociation constant KD.

The sixth and seventh steps 106, 107 can be determined in different ways according to different embodiments of the present invention. According to one embodiment, the sixth step 106 is performed through iteration where in each step the dissociation constant KD corresponding to the highest concentration of the equilibrium response sequence Rseq is determined based on the equilibrium response sequence Rseq, and the determined dissociation constant KD may then be associated with that concentration. Then, the highest value of the equilibrium response sequence Rseq is removed and the remaining values of the equilibrium response sequence Rseq is used for next step of the iteration.

Thus, the process of determining a dissociation constant KD based on the equilibrium response sequence Rseq and then removing one value from the sequence Rseq is repeated until the dissociation constants KD converges towards a value. This is determined through deciding a convergence value and comparing the difference between a newly determined dissociation constant KD and the one determined in the previous iteration. When said difference is smaller than the convergence value, the iteration is stopped and the latest dissociation constant is selected as the theoretical dissociation constant at zero concentration KD0.

According to another embodiment the theoretical dissociation constant at zero concentration KD0 is determined by collecting all the values for the dissociation constant KD obtained according to the previous embodiment described above and to plot them against the concentration and fitting a curve to the plot. From the fitted curve, the theoretical dissociation constant KD0 can be obtained by using the value of the curve when the concentration is zero.

As an alternative to determining each value for the dissociation constant KD based on the entire equilibrium response sequence Rseq, one value at a time from the equilibrium response sequence Rseq can be selected and an interval determined on each side of said value. The dissociation constant KD is then determined based on the value of the equilibrium response R at that value and a vicinity of the value.

In order to perform the steps of the method, it is advantageous to use software configured to perform the steps of the method, and to provide a computer readable medium configured to store the software. The computer readable medium can be a hard drive, an USB, a CD, among others.

### EXAMPLE

The use of the method will now be described with reference to an example. Using a surface plasmon resonance apparatus, KM01757 a small molecule with molecular weight 283.3 Da was used as analyte binding to immobilized Carbonic anhydrase II, a protein with a molecular weight of 29 kDa. The theoretical value for the equilibrium response Rmax was determined based on knowledge of immobilization level and molecular weights of the molecules and was 44 RU. On the sensor surface of the apparatus, the ligand was immobilized and a dilution series was created by using a plurality of samples having different, well defined concentrations of the analyte KM01757 in turn to contact the sensor surface, starting with the lowest concentration and proceeding towards the highest concentration. The concentrations and corresponding response values are shown by Table 1 below and can also be seen in FIG. 2 and FIG. 3.

TABLE 1
Concentrations of samples and corresponding
response values using SPR.

| Concentration μM | Response RU |
|---|---|
| 0 | 0 |
| 78 | 2.5 |
| 156 | 4.7 |
| 312 | 8.4 |
| 625 | 14.9 |
| 1250 | 25.6 |
| 2500 | 41.3 |
| 5000 | 63.4 |

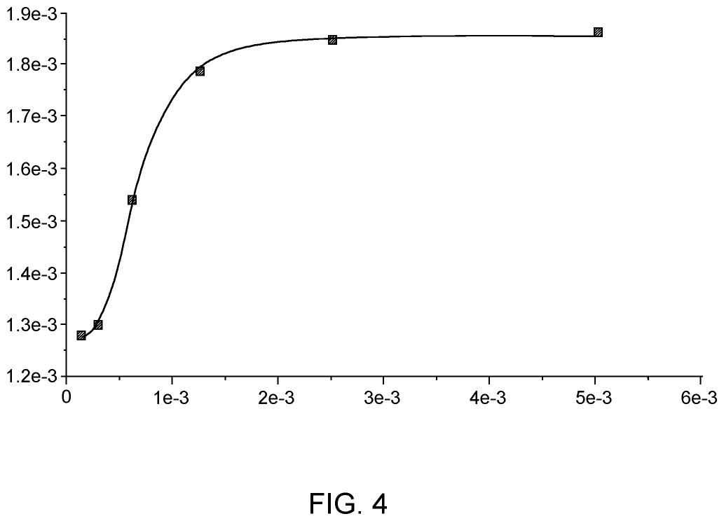

An equilibrium response sequence Rseq was created comprising the responses in Table 1 and the theoretical saturation response value Rmax. The dissociation constants KD were then determined based on the sequence Rseq and an iteration where one value at a time were removed from the sequence starting with the largest. Each dissociation constant KD was stored for later use. The data in FIG. 4 was obtained by fitting the data to Eq 1 above with Rmax1 set to 44 RU.

When less than three values were left in the sequence, the iterations were stopped, and the dissociation constants KD plotted against the concentration and a curve fitting performed, in this case the 4-parameter equation:

y=f(C)=Rhi−(Rhi−Rlo)/(1+((C/A1)\hat{ }A2)).

From the resulting curve, the value for the dissociation constant KD for zero concentration was obtained from the Rlo value and was 1.23 mM.

The invention is not to be seen as limited by the embodiments and the example described herein, but can be varied within the scope of the appended claims as will be readily apparent to the person skilled in the art. For instance, the method according to the invention is not limited to SPR but can be used with other technologies capable of providing steady state data such as biolayer-interferometry, calorimetry or thermophoresis, among others, and with different molecules. It should also be noted that each embodiment described herein can be freely combined with other embodiments if the person skilled in the art should so wish.

1. A method for determining affinity of a biomolecule, characterised by the steps of:
determining a theoretical saturation response value (Rmax) based on known properties of the biomolecule or a similar biomolecule;
providing a sensor surface having the biomolecule immobilized thereon as a ligand;
contacting the sensor surface with a plurality of samples containing different concentrations of an analyte that is able to bind to the ligand;
registering an equilibrium response (R) from binding of the analyte to binding sites of the ligand for each of the plurality of samples, said sensor response giving equilibrium response values for each sample;
creating an equilibrium response sequence comprising the determined equilibrium response values;
determining a plurality of values for a dissociation constant for the plurality of concentrations based on said equilibrium response sequence; and
determining a theoretical dissociation constant at zero concentration based on the plurality of values for the dissociation constant.

2. The method according to claim 1, wherein determining a plurality of values for a dissociation constant and the theoretical dissociation constant at zero concentration is performed by:
determining the dissociation constant corresponding to the highest concentration of the equilibrium response sequence based on the equilibrium response sequence;
selecting the equilibrium response value of the equilibrium response sequence corresponding to the highest concentration and removing said equilibrium response value;
determining the dissociation constant corresponding to the highest remaining concentration of the equilibrium response sequence and again removing the value corresponding to the highest concentration;
repeating the process of determining dissociation constant and removing a value from the equilibrium response sequence until the difference between the determined dissociation constant and the dissociation constant determined in the latest repetition is smaller than a convergence value; and
selecting the most recently determined dissociation constant as the theoretical dissociation constant at zero concentration.

3. The method according to claim 1, wherein determining a plurality of values for a dissociation constant and the theoretical dissociation constant at zero concentration is performed by:
determining the dissociation constant corresponding to the highest concentration of the equilibrium response sequence based on the equilibrium response sequence;
selecting the equilibrium response value of the equilibrium response sequence corresponding to the highest concentration and removing said equilibrium response value;
determining the dissociation constant corresponding to the highest remaining concentration of the equilibrium response sequence and again removing the value corresponding to the highest concentration;
repeating the process of determining dissociation constant and removing a value from the equilibrium response sequence until less than three values remain in the equilibrium response sequence;
plotting each determined dissociation constant against concentration to create a plurality of data points and fitting said data points to a curve; and
selecting a value along said curve corresponding to zero concentration as the theoretical dissociation constant at zero concentration.

4. The method according to claim 1, wherein each of the plurality of values for a dissociation constant is determined by selecting a value from the equilibrium response sequence, determining an interval on each side of the value and determining the dissociation constant based on said value and said interval.

5. Computer implemented software configured to perform the steps of the method according to claim 1.

6. A computer readable data storage medium holding software which is operable to perform any one of the methods according to claim 1.
